(19) United States
(12) Patent Application Publication
(10) Pub. No.: US 2016/0019374 A1
(43) Pub. Date: Jan. 21, 2016

(54) METHODS FOR ESTABLISHING COMBINATIONS OF KINASE INHIBITORS FOR THE TREATMENT OF MEDICAL CONDITIONS

(71) Applicant: SALGOMED, INC., Del Mar, CA (US)

(72) Inventor: Edison Ong, Del Mar, CA (US)

(21) Appl. No.: 14/802,911

(22) Filed: Jul. 17, 2015

Related U.S. Application Data
(60) Provisional application No. 62/026,110, filed on Jul. 18, 2014.

(51) Int. Cl. G06F 19/00; G01N 33/50

(52) U.S. Cl. CPC G06F 19/706; G01N 33/5011; G06F 19/704; G06F 19/705; G01N 2500/10

(57) ABSTRACT

Methods that incorporate drug libraries and in vitro measurements to predict the response of cells to previously untested drug combinations containing two or more compounds to identify drug combinations for use as a pharmaceutical in the treatment of cancers and other disease using regression analysis of cell-based assays.

### CROSS REFERENCE TO RELATED APPLICATIONS

This invention claims benefit of priority to U.S. provisional patent application Ser. No. 62/026,110 filed Jul. 18, 2014; the entire content of which is herein incorporated by reference in its entirety.

### TECHNICAL FIELD

The invention relates to methods for predicting computationally the sensitivity of cells to combination of compound molecules that modulate a set of endogenous target molecules to produce a beneficial biomedical or biological effect.

### BACKGROUND ART

Statistical inference and regression methods in conjunction with gene expression or mutations have been used to identify specific biomarkers associated with an increased sensitivity/resistance to drugs and to predict drug sensitivity. For instance, the sensitivity to PARP inhibitors of Ewing's sarcoma cells with mutations in the EWS gene and to MEK inhibitors in NRAS-mutant cell lines with AHR expression have been predicted using analysis of variance and the elastic net method¹ and then experimentally validated.^{2,3} In these analyses, the statistical variable associated to drugs was represented by the half maximal inhibitory concentration (IC50) in different cell lines. However, besides the IC50, there are many other types of information that characterize chemical compounds. These types of information can enhance the statistical analyses and improve the accuracy of predictions. For instance, a method to predict drugs sensitivity in cell lines based on the integration of genomic data with molecular physico-chemical descriptors of the drugs has been recently proposed.⁴ Another useful type of information is the residual activity of drug target proteins after interacting with a compound. This information is often available for drugs belonging to the class of kinase inhibitors. The important role of kinases in cancer biology⁵ has spurred a considerable effort towards the synthesis of libraries of fully profiled kinase inhibitors, providing a map of the strength of each compound on a large number of its drug targets.⁶⁻⁸ A recently published dataset has profiled several hundred kinase inhibitors using a panel of more than 300 kinases.⁸ Kinase profiling, patient genetic profiles, and sensitivity of primary leukemia patient samples to kinase inhibitors were recently used by Tyner et al.⁹ to identify functionally important kinase targets and clarify kinase pathway dependence in cancer.

### SUMMARY OF THE INVENTION

The invention provides a computational method that incorporates profiled drug libraries and in vitro measurements to predict the response of cells to previously untested drug combinations containing two or more compounds. Besides making prediction about the cellular response to drugs, the method identifies critical drug targets and pathways that are statistically associated to drug sensitivity in a given cell line. To this end the invention also provides drug combinations containing two or more compounds identified using the methods herein together with a pharmaceutically acceptable carrier to form a pharmaceutical for the treatment of disease.

The above is accomplished through a method of quantitatively predicting effectiveness of drug combinations with two or more compounds for use as a therapy for a disease. In Some embodiments the disease is a cancer, such as breast cancer, lung cancer, colon cancer, prostate cancer, melanoma, or other cancer identified by the National Cancer Institute (NCI). The method includes providing a biological sample from a patient suffering from a disease, optionally a cancer, the sample comprising cells from the patient; providing a library of drugs in which each drug i has known residual activity A_{k,i} of each drug-target protein k under the effect of the drug i; assaying the cells from the biological sample with each drug i to obtain a viability parameter vᵢ for each drug, wherein the assay is an endpoint assay that determines growth, survival, or death of a living cell; performing a regression analysis using results from the assays to determine a training set and to identify residual activity parameters as predictors of the viability, wherein the results are modeled assuming a dependence as Equation (I):

$${v}_{i} = {e}^{{β}_{0}} {\prod }_{k = 1}^{p} {( {A}_{k , i} )}^{{β}_{p}} \qquad ( I )$$

and the regression analysis provides a set of coefficients β₀, . . . , βₚ, where p is a number of drug-target proteins; predicting the viability of a combination COMB with N drugs and dosages d=(d₁, d₂, . . . , d_{N}) as Equation (II)

$${v}_{COMB} = {e}^{{β}_{0 }} {\prod }_{k = 1}^{p} {( {A}_{k , COMB} )}^{{β}_{p}} \qquad ( II )$$

where residual activity A_{k,COMB} of the drug target k under the effect of a combination with N drugs and dosages d=(d₁, d₂, . . . , d_{N}) is calculated according to Equation (III)

$${A}_{k , COMB} = {\prod }_{j = 1}^{N} {( {A}_{k , j} )}^{{d}_{j}} \qquad ( III )$$

and searching within the space of dosages d=(d₁, d₂, . . . , d_{N})to find a corresponding optimal value of the predicted combination viability v_{COMB}; and selecting the identified optimal combination of drugs as a combinatorial therapy for a patient having a same cancer or same disease.

In some embodiments, the Equation (I)

$${v}_{i} = {e}^{{β}_{0}} {\prod }_{k = 1}^{p} {( {A}_{k , i} )}^{{β}_{p}} \qquad ( I )$$

is reduced to a linear form using a logarithmic transformation and the coefficients (β₀, β₁, . . . , βₚ) are obtained using a linear regression procedure including the lasso, ridge, or elastic net methods.

In some embodiments the biological sample is a bodily fluid, such as blood or serum; however, in other embodiments the biological sample is a tumor or bodily tissue, such as a biopsied tissue from any organ. The skilled artisan will appreciate that cells, such as cancer cells, may be harvested from the biological sample using any suitable approach. Examples of cancer cells and thus cancers that can be treated using the methods of the invention include breast cancer cells, lung cancer cells, prostate cancer cells, melanoma cancer cells and other cancer cells identified by the National Cancer Institute (NCI). In other embodiments the cells may be non-cancerous cells from different tissues for predicting toxicity of a combination of two or more compounds.

In some embodiments the predictions of viability and toxicity are combined in a prediction or form a therapeutic index of a drug combination with two or more compounds. The therapeutic index of different drug combinations can then be compared and desired combinations chosen for combination with a pharmaceutically acceptable carrier to form a pharmaceutical as known in the medical arts. In some embodiments the predictions of viability are combined to predict synergistic and antagonistic properties of drug combinations for potential therapy. Once the pharmaceutical is formed it can be administered to the same patient from which the biological sample was taken or can be administered to a different patient suffering from the same disease or cancer. In some instances a pharmaceutical formed from a combination of two or more compounds selected from assays performed on cells obtained from a first cancer sample is selected for the treatment of a patient suffering from a different cancer.

In some embodiments the magnitude of the coefficients β₁, . . . , βₚ is used to identify drug-target proteins whose inhibition or stimulation is associated to a positive therapeutic response.

In some embodiments the library of drugs consists of compounds in the drug class of kinase inhibitors.

### BRIEF DESCRIPTION OF THE DRAWINGS

FIG. 1 is a chart providing a prediction of combination effects. In particular, a comparison between combinatory predictions obtained using the invention and experimental results are shown. Each dot corresponds to the myoblast viability obtained from 72 combinations between four drugs: G13, O20, I15, and K10 (R2=0.70, P<0.00001).

FIG. 2 is a graph of primary screen results of the top ten most selective kinase inhibitors. Drugs are ranked based on the IMR-90 to A549 viability ratio. The 3 digit codes identify the compounds: A15: PDK1/Akt1/Flt3 Dual Pathway Inhibitor (CAS 331253-86-2); E20: Cdk/Crk In. (CAS 784211-09-2); O20: SU9516 (CAS 666837-93-0); H15: MEK1/2 In. II (CAS 212631-61-3); L13: PI 3-Kα In. VIII (CAS 372196-77-5); G10: Fascaplysin, Synthetic (CAS 114719-57-2); D07: Cdk2 In. II (CAS 222035-13-4); C16: Cdk1/2 In. III (CAS 443798-55-8); M16: GSK3b In. XII, TWS119 (CAS 601514-19-6); N05: Reversine (CAS 656820-32-5).

FIG. 3 is a graph showing a dose response curve of PDK1/Akt1/Flt3 Dual Pathway Inhibitor. Different doses of PDK1/Akt1/Flt3 Dual Pathway Inhibitor were tested to measure the response of A549 to the drug. For the secondary screen we selected 125 nM to ensure low toxicity on the normal cell line.

FIG. 4 is a graph depicting secondary screen results of the top ten most selective drugs (1000 nM) when paired with PDK1/Akt1/Flt3 Dual Pathway Inhibitor at 125 nM. Selectivity is the IMR-90 to A549 viability ratio. The 3 digit codes identify the compounds: A12: Alsterpaullone, 2-Cyanoethyl (CAS 852529-97-0); D17: Cdk2/9 In (CAS 507487-89-0); K08: K-252a, Nocardiopsis sp. (CAS 97161-97-2); O21: Staurosporine, Streptomyces sp. (CAS 62996-74-1); P15: WHI-P180, Hydrochloride (CAS 211555-08-7); E13: Gö 6976 (CAS 136194-77-9); C09: Compound 56 (CAS 171745-13-4); A10: Alsterpaullone (CAS 237430-03-4); O03: AG 1478, Selective inhibitor of epidermal growth factor receptor (EGFR) protein (CAS 175178-82-2); N05: Reversine (CAS 656820-32-5).

FIG. 5 is a set of charts showing a Leave-one-out Cross Validation of the elastic net regression model based on the primary (top) and secondary (bottom) screens for normal and cancer cell lines. Each of the 140 points in these figures corresponds to one of the 140 drugs. “Regression” refers to the viability predicted by the regression model using all data from the other 139 drugs as training set, while “Measured” refers to the actual viability measured for the drug or drug combination. Note that only the secondary screen leads to predictive models with significant R2 for the two cancer cell types.

FIG. 6 is a set of charts showing Leave-one-out Cross Validation of the elastic net regression model based on the primary (top) and secondary (bottom) screens for normal and cancer cell lines after logarithmic transformation on the data. Each of the 140 points in these figures corresponds to one of the 140 drugs. “Regression” refers to −log of the viability predicted by the regression model using all data from the other 139 drugs as training set, while “Measured” refers to −log of the actual viability measured for the drug or drug combination. Note that, as in FIG. 4, only the secondary screen leads to predictive models with significant R2 for both cell types. The R2 for the Cancer cell lines is considerably better using the log transformation.

FIG. 7 is a table of correlations between selectivity and kinase activity from primary and secondary screening. A negative correlation indicates that inhibition of that particular kinases is associated to a higher selectivity. The top two hits with negative correlation, TGFBR2 and CDK4 are known to have an important role in cell proliferation, invasion and metastasis in lung adenocarcinoma^{10,11}.

FIG. 8 is a table of kinases with the highest difference in the regression coefficients for the log transformed data of the secondary screen. A larger difference is associated with a selective response of A549 upon inhibition. Note that in addition to TGFB2R and CDK4, which were identified with the correlation approach of Table 1 (FIG. 7), additional kinases known to have an important role in lung cancer such as EGFR^{12,13} and PHKG1¹⁴ are found using the elastic net approach.

FIG. 9 is a table of reactome pathways with significant representation of kinases from the regression analysis. Ns indicates the number of kinases that are found significant in the regression analysis, while NT is the total number of kinases in the pathway. The top ten pathways with Fisher exact test p<=0.051 are shown. These pathways are identified from 518 Reactome pathways containing at least one of the kinases identified in Table 2 (FIG. 8). The 9 kinases in the axon-guidance pathway are EGFR, PAK1, ERBB2, CDK5, GSK3B, PAK2, RPS6KA2, FGFR1 and PAK7.

### DETAILED DESCRIPTION

For clarity of disclosure, and not by way of limitation, the invention is discussed according to different detailed embodiments; however, the skilled artisan would recognize that features of one embodiment can be combined with other embodiments and is therefore within the intended scope of the invention.

Unless defined otherwise, all technical and scientific terms used herein have the same meaning as is commonly understood by one of ordinary skill in the art to which this invention belongs. If a definition set forth in this document is contrary to or otherwise inconsistent with a well accepted definition set forth in the art, the definition set forth in this document prevails over a contradictory definition.

The term “residual activity” as used herein refers to the activity of a protein after inhibition by one or more drugs, e. g, the activity of a kinase after inhibition by one or more kinase inhibitors.

The term “viability parameter” as used herein refers to a measure of viability of a cell population, usually expressed as a percentage of a control untreated population of cells.

The term “optimal value of a predicted combination viability” as used herein refers to either a maximum or a minimum of the viability parameter, depending on the specific therapeutic application.

The term “therapeutic index” or “therapeutic index of a drug” as used herein refers to ratio of the viability of the disease cells, e.g. cancer cells, over the viability of normal control cells treated with the same drug or drug combination.

The term “endpoint assay” as used herein refers to a measurement of growth, survival, or death of a cell at a specific therapeutically relevant time-point.

As an introduction to the invention we have introduced an integrated experimental and computational methodology that identifies the role of specific kinases in the drug response of a given cell line. The key element of our KIEN methodology is a multiple regression procedure that uses in vitro screen data as a training set. If a new library of kinase inhibitor compounds were to be synthetized and profiled, then our model would be able to immediately estimate the effect of these drugs on in vitro experiments on a given cell line. In support of our approach we show an application to a lung cancer cell line in Example 3, but the skilled artisan will appreciate that our method can be extended to different cancerous and non-cancerous cell lines. The method also facilitates the design of new kinase inhibitors and the development of therapeutic interventions with combinations of many inhibitors.³⁰ While in some embodiments at least two drugs or compounds for the combination are used, in other embodiments the procedure could be extended to three drug combinations, or more than three drug combinations. Finally, the method could be extended to regression models that are specific of cancer cells with the same set of mutations, or it could be directly used with patient-derived primary cells to identify a personalized treatment.

Drug-kinase profiling represents a controller-target network²⁰ that when combined with in vitro testing, can be used in regression models to predict drug response and to identify pathways statistically associated to drug sensitivity. Network methods in biology are often based on the analysis of large datasets from high-throughput experiments. An example is given by gene regulatory networks, which presents many challenges either when restricted to a homogeneous set of data^{21,22} or when it includes different classes of data.²³⁻²⁶ In our method, information from the drug-target network and experimental query of the biological system are integrated. The goal is not a reconstruction of a regulatory network, but to identify a set of kinases linked to a therapeutic response in a given cell line. In order to establish associations, the system has to be perturbed by the use of kinase inhibitor drugs. The response to these single drugs or drug combinations becomes a training set that when combined with the kinase profiling, can lead to predictions.

The elastic net method is one of the most widely used regularization techniques. Regularization techniques are used in statistical and machine learning models to achieve an optimal tradeoff between accuracy and simplicity. Simplicity makes a model less prone to overfitting and more likely to generalize. In our analysis, we found that the elastic net regressions based on single drug responses were not successful, while drug pair data provided statistically significant predictions. A possible explanation for this finding is the following: single drugs might be less able to overcome the robustness of biological networks.²⁰ The phenotypic signal is therefore blunted and not easily measured. If a second drug is added, any compensatory capacity is already stretched and the effects from the inhibition of each kinase can be seen more clearly. Using data from drug pairs, we found that noise can be better filtered out and stronger statistical associations between kinases and therapeutic response are revealed. Clearly, if a different training set with higher variance in efficacy measures were used in the primary screen, it is likely that also single drug in vitro response would have given a significant predictive model.

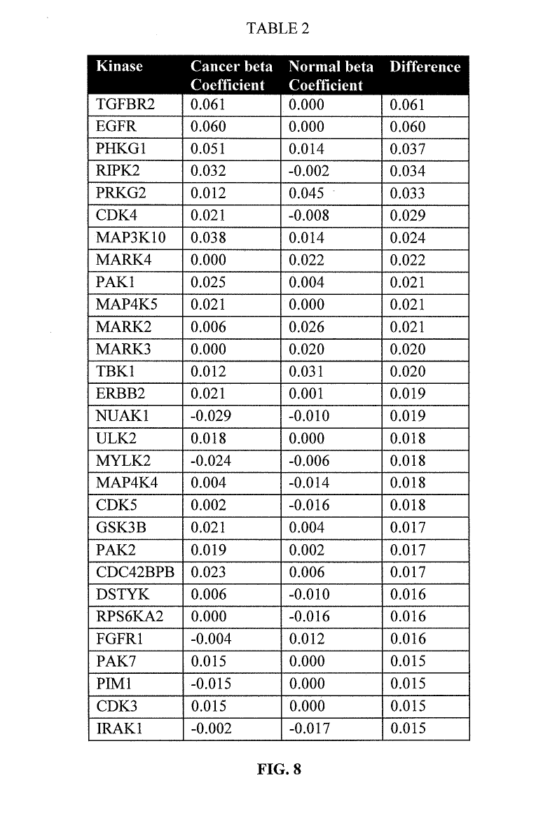

We identified several kinases that are implicated in lung cancer that gives biological significance to our KIEN method. In particular, TGFBR2 appears as a top hit both in the correlation and in the elastic net methods. This finding is consistent with recent siRNA experiments on A549 cell lines,¹⁰ which demonstrated that silencing of this receptor reduces cell proliferation, invasion, and metastasis. The Cyclin-dependent kinase 4 (CDK4) appears as a second top target in the correlation analysis, and is also highly significant in the KIEN analysis. Experiments using lentiviral-mediated shRNA to inhibit CDK4 in A549 have shown inhibited cell cycle progression, suppressed cell proliferation, colony formation, and migration,¹¹ and there is an ongoing clinical trial using a CDK4/6 inhibitor in lung cancer.²⁷ The KIEN analysis identified EGFR, which is known to be overexpressed in the majority of non-small cell lung cancers.¹² Furthermore, RNAi experiments targeting EGFR demonstrated cancer growth suppression in A549 xenograft in mice.¹³ The third kinase in Table 2 (FIG. 8), PHKG1 has also been found to be upregulated in human tumor samples, including lung adenocarcinoma, and aberrations in its gene copy number is a feature of many human tumors¹⁴.

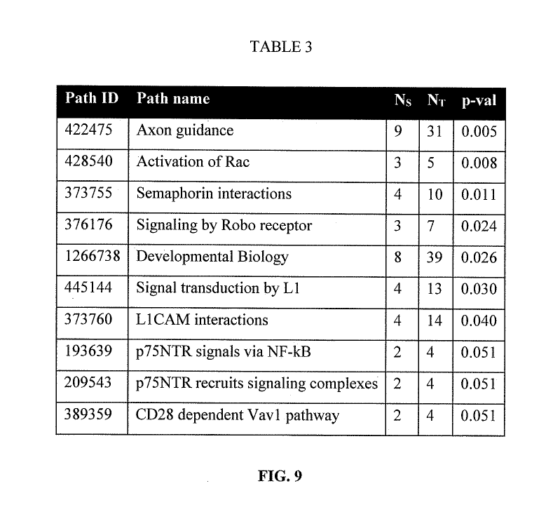

The pathway-based enrichment provides a broader view on the role of the kinases identified by our method in Table 2 (FIG. 8). Among the top three pathways shown in Table 3 (FIG. 9) are activation of Rac and Semaphorin interactions. Rac proteins play a key role in cancer signaling and they belong to the RAS superfamily.²⁸ We also identified a set of semaphorins in our analysis that is represented in the top significantly enriched pathways. Semaphorins, previously known as collapsins, are a set of proteins containing a 500-amino acid sema domain among others (including PSI and immunoglobulin type domains), which can be transmembranous or secreted.²⁹ It is known that Sema3E cleavage promotes invasive growth and metastasis in vivo.²⁹ These genes also have selective targeting by Rac and Rho family members. This generates hypotheses of possible pathways that could be targeted therapeutically. However, these hypotheses need to be validated by further experiments with different inhibitors for the same targets or with alternative methods, e.g. using siRNA.

### EXAMPLE 1

### Quantitatively Predicting Effectiveness of Drug Combinations with Two or more Compounds

Materials

The primary screening of a kinase inhibitor (KI) library comprised of 244 KIs was purchased from EMD Chemicals, and diluted with DMSO to 2 mM concentrations for high-throughput screening purposes. The KI library was stored at −80° C. Additionally, PDK1/Akt1/Flt3 Dual Pathway Inhibitor (CAS #331253-86-2) was ordered from EMD. Only 140 out of 244 were used in the drug-target network reconstruction because the drug profiling information was available only for these compounds. One kinase inhibitor known to affect the kinase targets indirectly was excluded.

Cell Culture

Cell lines IMR-90 (normal lung fibroblast) and A549 (lung adenocarcinoma) were cultured in RPMI 1640 (Hyclone) supplemented with 10% Canadian characterized fetal bovine serum (Hyclone), 1% 200 mM L-glutamine (Omega), and 1% penicillin/streptomycin (Omega). The media for the cells were renewed every 3 days and kept at 80-90% confluency. Cells were maintained in a humidified environment at 37° C. and 5% CO2.

Kinase Inhibitor Experiments

IMR-90 (1500 cells/well) and A549 (750 cells/well) were seeded on 384-well microplates (Grenier Bio-One) and incubated for 3 hours before the addition of kinase inhibitor(s). The reason that IMR-90 was seeded at double the cell density of A549 is due to the difference in cell division. IMR-90's doubling time is 36-48 hours whereas A549's is 22 hours. We wanted to make sure that the cells have divided at least once during the 72 hr drug treatment. Furthermore, both A549's and IMR-90's final confluency at 72 hrs is 90-95% and within the range of the ATPlite 1 step assay. ECHO 555 Liquid Handler (Labcyte) was used to dispense nanoliter volumes of each KI to 384-well plates with cells attached (wet dispense). The final volume in the plate is 40 uL and cells were incubated for 72 hours with KI treatment.

ATP Measurements

ATPlite 1 Step (Perkin Elmer) was used to evaluate the cell number and cytotoxicity. ATP measurements were done by dispensing 20 uL of the ATPlite 1 Step solution to each well to a final volume of 60 uL. The plate was placed on a shaker at 1100 rpm and the luminescence activity was detected by Analyst GT Plate Reader. The percent (%) of control is the quantity of ATPlite 1 step measurement of the treated versus the untreated wells of each individual cell type.

The ATP standard was prepared with culture media to final volume of 40 uL, and 20 uL of ATPlite 1 step reagent was added.

Computational Methods

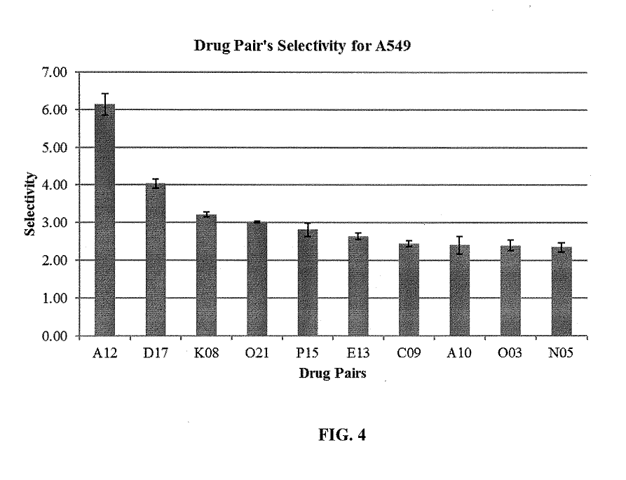

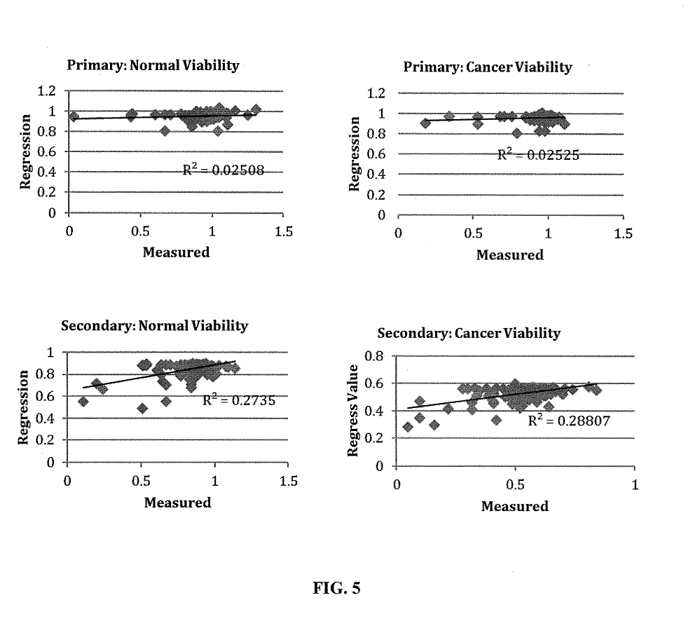

Correlations between selectivity/viability and kinase activity were calculated using the python scipy linregress function, which also provide p-values. Ranking the p-values and directly applying the Benjamini-Hochberg procedure gave us the FDR values. The elastic net regression was carried out using the Scikit-learn package³¹ which finds the coefficients β that minimize the function according to Equation (IV):

$$F = \frac{1}{2 M} {‖ v - A β ‖}_{2}^{2} + α ρ {‖ β ‖}_{1} + \frac{1}{2} α ( 1 - ρ ) {‖ β ‖}_{2}^{2} \qquad ( IV )$$

where v is the vector of the observed viabilities and A is the matrix containing the residual activity of the kinases from the profiling, and M is the total number of drugs or drug combinations used. The parameters α and β determine the relative weights of the lasso and ridge penalties quantified using Lⁱ (∥·∥₁) and L² (∥·∥₂) norm, respectively. We used α=0.15 and p=0.01 in the results of FIGS. 4 and 5 and in Table 2 (FIG. 8). We also tried other values of these parameters, which did not give a significant difference in the results.

Pathway-Based Enrichment

Reactome pathways were downloaded using a newer build of the ‘biomaRt’ library (v2.12.0) in Bioconductor/R (v2.15.0). Gene symbols from the kinase list were converted to Entrez gene identifier numbers (‘entrezgene’) and mapped against the gene ids in each Reactome pathway. For each pathway, the set of significant genes enriched within any given pathway was computed using a Fisher exact test. The procedure computes the significance (p-value) of observing significant kinases, as deemed significant by our method, within the selected pathway. These pathways are identified from 518 Reactome pathways. Given that our gene set consists entirely of kinases and would be generalized towards kinase-specific effects, the set of all kinases (˜300) were selected for background adjustment and more sensitive enrichment of the pathways. This procedure was repeated for each pathway to generate p-values and pathway rankings. False discovery rate [FDR] values were later generated to further restrict significance.

### EXAMPLE 2

### Quantitatively Predicting Effectiveness of Drug Combinations with Two or more Compounds with Application to Hypoxia

We used our method to predict synergistic effects between four kinase inhibitors: G13, O20, I15, and K10. These kinase inhibitors were selected for their ability to protect primary myoblasts against 0.1% hypoxia. Out of 244 drugs from the EMD library of kinase inhibitors, we first selected 58 drugs that have a positive effect on cell survivability against hypoxia. Single-drug screening results with these drugs were used as a training set to build a regression model that uses the kinase catalytic activity as predictor of the viability. The model assumed a dependence of the viability Vᵢᵢ of the form of Equation (V)

vᵢ=e^{β}^{₀}*(A_{1,i})^{β}^{₁}* . . . *(A_{p,i})^{β}^{ₚ} (V)

where we indicate as A_{k,i} the residual activity of the kinase k under the effect of drug i. The residual activities were obtained from a published dataset containing catalytic activities of kinase inhibitor targets⁸. P17 was also found to protect primary myoblasts, but was excluded in our analysis since the kinase profiling information was not available in this dataset.

Equation (V) can be reduced to a linear form using a logarithmic transformation, and the coefficients (β₀, β₁, . . . , βₚ) can obtained using a linear regression procedure. The resulting coefficients βₖ can be interpreted as a measure of the protective impact of kinase k on the cell survival against hypoxia. A higher value of the coefficient of a kinase indicates a higher protective effect of that kinase on the survivability.

The coefficients βₖ were then used to predict the viability of four-drug combinations. We assumed that the residual activity A_{k,c} of the kinase k under the effect of a four-drug combination c with dosage d=(d₁, d₂, d₃, d₄) is

A_{k,0}=A_{k,1}^{d}^{₁}*A_{k,2}^{d}^{₂}*A_{k,3}^{d}^{₃}*A_{k,4}^{d}^{₄}. (VI)

The predicted viability of a four-drug combination, vₐ can then be obtained using a modified form of Equation (V) as

v_{c}=e^{β}^{₀}*A_{1,c}^{β}^{₁}* . . . *A_{p,c}^{↑}^{_{P}}, (VI)

where the coefficients βₖ are determined using the linear regression on the single-drug data. In the prediction we only considered kinases with positive coefficients since we are modeling a protective effect on cell survival against hypoxia.

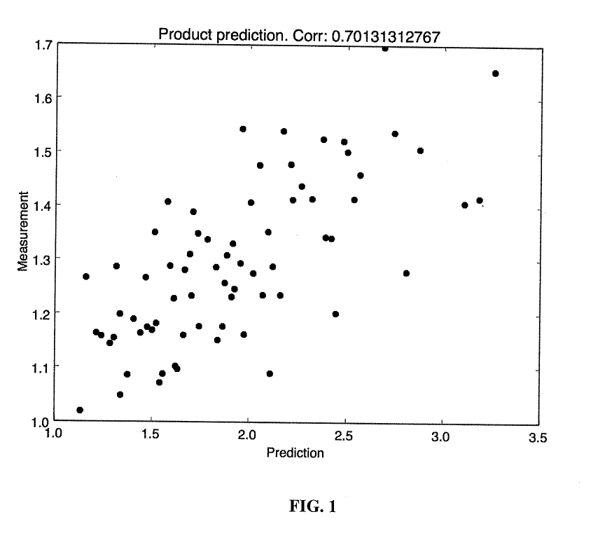

FIG. 1 shows predictions obtained with the method above and the corresponding experimental results. Each of the 72 points indicates a combination of 4 drugs (G13, O20, I15, and K10) with different dosages. The Person correlation between the predicted and measured values is 0.70, indicating that the prediction with the method is significant.

The results in FIG. 1 show that our invention can predict which combinations of kinases will be effective for hypoxia protection given information from the screen of a library of single drugs. Large libraries of characterized kinase inhibitors are available, at least to large pharma companies, and this method might make the search for effective kinase inhibitor combinations more efficient. The coefficients of the model indicate the most important kinases responsible for the protective effect. We might therefore develop a combination of kinase inhibitors targeting not a single kinase but a set of kinases with the appropriate quantitative amount of inhibition.

### EXAMPLE 3

### Quantitatively Predicting Effectiveness of Drug Combinations with Two or more Compounds for Lung Cancer

In Vitro Screen of the Kinase Inhibitor Library

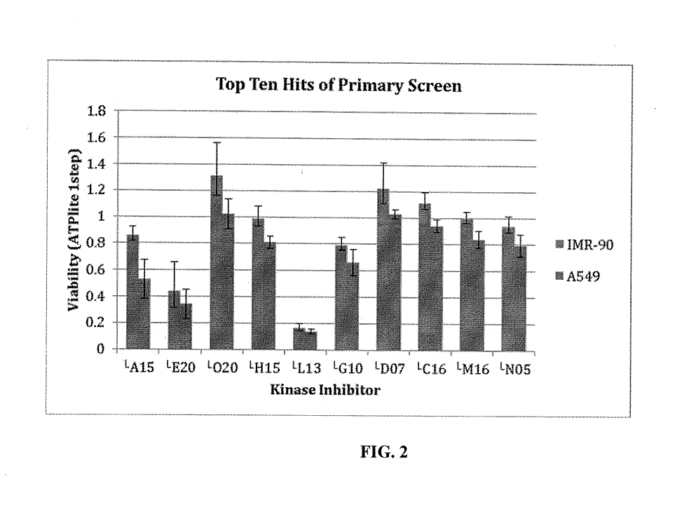

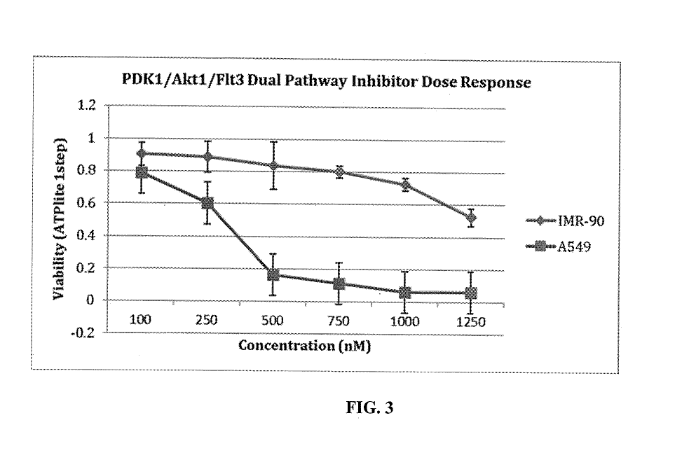

Our methodology begins with the high-throughput screening of single drug and drug pair experiments. The 244 kinase inhibitors (KIs) of the EMD drug library were screened at 1000 nM individually and the treatment lasted for 72 hours. To quantify a selective response of a cancer cell line with respect to a control normal cell line, we define the selectivity S of a single drug or drug combination as

$$S = \frac{{v}_{N}}{{v}_{C} } \qquad ( VII )$$

where v_{N} indicates the viability of normal cells (IMR90) after treatment, and v_{c} the viability of cancer cells (A549) after treatment. From the screening of the 244 KIs, the top hit was PDK1/Akt1/Flt3 Dual Pathway Inhibitor (CAS #331253-86-2) as ranked by selectivity (FIG. 2). For the secondary screen, we used the PDK1/Akt1/Flt3 Dual Pathway Inhibitor as the starting point and combined this compound with the other KIs as a drug pair combination. The dose of PDK1/Akt1/Flt3 Dual Pathway Inhibitor was studied to ensure proper dosing range and minimize toxicity. We used 125 nM, which maintains the normal cell line IMR-90's viability >90% (FIG. 3). For the other 243 KIs we used the standard dose of 1000 nM. Several pairs in the secondary screen showed very high selectivity. The top hit from the secondary screen of the library was Alsterpaullone 2-cyanoethyl (CAS #852529-97-0) with a selectivity of S=6.14 for the pair (FIG. 4).

Analysis of Correlations

In our second step, we analyzed the Pearson's correlation of the primary and secondary screening with a published dataset¹⁵ containing target profiles for 140 kinase inhibitors. Therefore, even though we had a library of 244 KIs in the experimental screening, we were limited to utilizing 140 KIs for the analysis. For each inhibitor, the dataset provides the residual activity (0≦A≦1) of 291 kinases after drug treatment. This quantity is a measure of the strength of inhibition of a drug on each kinase.

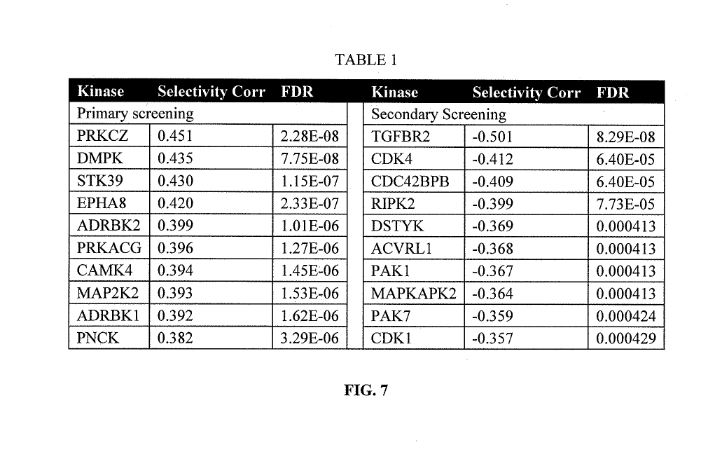

For each kinase k, we calculate the Pearson's correlation, Cₖ, between the selectivity Sᵢ and the activities A_{k,i}, with i ε {1, . . . , M} indicating the single drug or drug pair in the set. For drug pairs, the activity is estimated as a product of the residual activities of the two drugs. The kinases are then ranked based on the p-value of their correlation with selectivity, and we calculate the False Discovery Rate (FDR) adjusted p value.¹⁶ The list of kinases mostly correlated to the selectivity from the primary and secondary screen are listed in Table 1 (FIG.7). We also calculated the correlation between the normal or cancer cell viability and the activities.

Elastic Net Regression

Next, we built a regression model that predicts the response of a cell line to a drug or drug combination i. The response we predict is the normal and cancer cell viability, from which the selectivity can be derived. For this purpose, we define a regression problem in which we use the residual activity of the kinase k under the effect of drug i, which we indicate as A_{k,i}, as predictors of the viability. The response can be written as Equation (VIII)

vᵢ=β₀+β₁A_{1,i}+ . . . +βₚA_{p,i}. (VIII)

A fitting procedure based on a training set of measurements produces the coefficients (β₀, β₁, . . . , βₚ). Equation (VIII) can then be used to predict the viability of a new drug that has not been tested, but of which the profiling information is available. Note that we are integrating two different types of data: kinase profiling data is obtained through enzymatic assays that probe directly the interaction between drug and kinases, while the in vitro cell response data is the result of complex signaling that involves many pathways downstream of the affected kinases. The coefficients βₖ can be seen as a measure of the sensitivity of a given cell line due to alterations in the activity of kinase k.

It is well known that the least square method does not perform well in the case of linear regression with many predictors. In our case, we would like to use a database of drugs that have been profiled on about 300 kinases. However, it would be desirable to select and keep in the final model a minimal set of the kinases that provide a simple model, useful to gain biological insight. The lasso technique¹⁷ is a powerful method to reduce the number of predictors by imposing a penalty on the regression coefficients. However, in the presence of a group of kinase predictors with strong mutual correlation, the lasso could select only one kinase predictor from the group while missing the others. To prevent this problem, our method uses the elastic net approach. This method incorporates the lasso penalty as well as a ridge penalty to keep the regression coefficients small without completely removing them.¹⁸ The weights of the ridge and lasso penalties in the least square procedure can be optimized for best performance of the method.

We show in FIGS. 5(a) and (b) the results of a leave one out cross validation (LOOCV) method for the primary (a) and secondary screen (b). For each of the 140 drugs, we apply the elastic net method using the remaining 139 drugs and then we compare the result to the measured value. This cross validation method is a particular case of the more general k-fold cross validation procedure in which k is equal to the size of the training set.¹⁹ The cross LOOCV shows that the information contained in the primary screen is not sufficient to define a predictive model. The fact that some kinases in Table 1 (FIG. 7) show some significant correlation with the response when considered individually is in general not a sufficient condition for defining a predictive, multiple regression model. On the other hand, the secondary screen is able to reproduce the viability of many drugs, especially the ones with the stronger effect on both cell lines. Overall, the data from the secondary screen presents a much broader distribution with a tail representing a few drug combinations particularly effective. The regression works better in identifying these highly effective pairwise combinations and the relative ranking of their strengths. Data is not particularly informative for drugs and drug pair combinations that are not effective, which concentrate in the neighborhood of ˜1.

Data transformations can represent a powerful strategy to improve regression. We applied a logarithmic transformation, which is consistent with the hypothesis of an independent action on the different kinases on the total viability. In this case we assume that the viability can be rewritten in the form of Equation (IX)

v₁=e^{β}^{₀}(A_{1,1})^{β}^{₁}·(A_{2,1})^{β}^{₂}· . . . ·(A_{p,1})^{β}^{ₚ}. (IX)

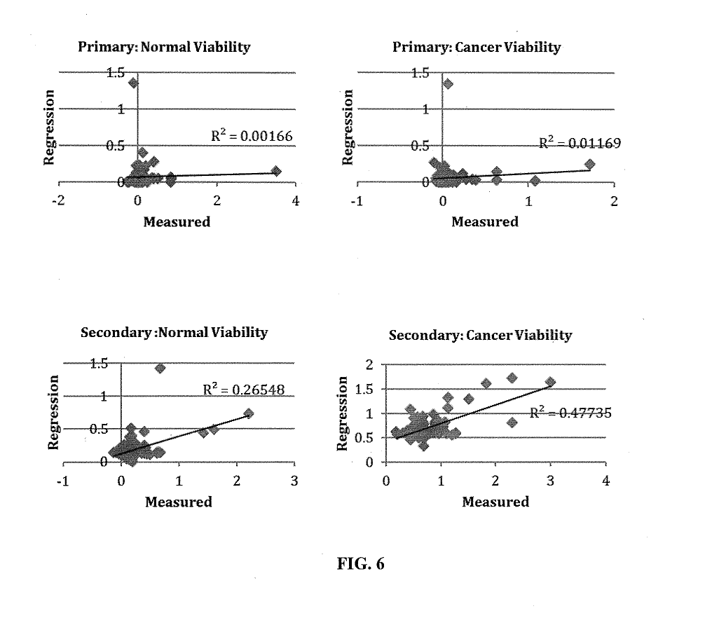

By applying a log transformation on both sides of Eq. (IX) we reduce the problem to a linear regression, to which the elastic net strategy can be applied. We show in FIG. 6 the results of the LOOCV for the primary and secondary screen using the logarithmic data transformation. As in the linear case, we find that the method fails the cross validation procedure if we use data from the primary screen, while the secondary screen with log transformed data gives better R2.

In addition to a regression model that can be used to predict the efficacy of drugs that have not been tested, the βᵢ can be used to rank kinases in terms of their relevance in the regression. Therefore, these coefficients identify the kinases whose inhibition is associated to a decrease in the cell viability. A ranking based on the differential βᵢ^{C}−βᵢ^{N}, where the index N and C identify the regression model of the cancer and normal cells, gives insight on specific pathways important for a selective response of cancer cells. Table 2 (FIG. 8) shows a list of kinases ranked in terms of |βᵢ^{C}−βᵢ^{N}|, where the coefficients have been obtained using the logarithmic data transformation on the secondary screen.

In order to test whether selected pathways were significantly enriched for the identified kinase genes in Table 2 (FIG. 8), a pathway-based enrichment analysis was conducted using the results from the elastic net kinase analysis and Fisher exact tests. Ten pathways from Reactome were identified as significant (p<0.05) using this kinase list, including axon guidance, activation of Rac, and semaphorin interactions as top hits (Table 3 (FIG. 9)).

The invention described herein may be embodied in other specific forms without departing from the spirit or essential characteristics thereof. The specific embodiments previously described are therefor to be considered as illustrative of, and not limiting, the scope of the invention.

### REFERENCES

1 Zou, H. & Hastie, T. Regularization and variable selection via the elastic net. J. R. Statist. Soc. B 67, 301-320, (2005).

2 Garnett, M. J., Edelman, E. J., Heidorn, S. J., Greenman, C. D., Dastur, A., Lau, K. W., Greninger, P., Thompson, I. R., Luo, X., Soares, J., Liu, Q., Iorio, F., Surdez, D., Chen, L., Milano, R. J., Bignell, G. R., Tam, A. T., Davies, H., Stevenson, J. A., Barthorpe, S., Lutz, S. R., Kogera, F., Lawrence, K., McLaren-Douglas, A., Mitropoulos, X., Mironenko, T., Thi, H., Richardson, L., Zhou, W., Jewitt, F., Zhang, T., O/'Brien, P., Boisvert, J. L., Price, S., Hur, W., Yang, W., Deng, X., Butler, A., Choi, H. G., Chang, J. W., Baselga, J., Stamenkovic, I., Engelman, J. A., Sharma, S. V., Delattre, O., Saez-Rodriguez, J., Gray, N. S., Settleman, J., Futreal, P. A., Haber, D. A., Stratton, M. R., Ramaswamy, S., McDermott, U. & Benes, C. H. Systematic identification of genomic markers of drug sensitivity in cancer cells. Nature 483, 570-575, (2012).

3 Barretina, J., Caponigro, G., Stransky, N., Venkatesan, K., Margolin, A. A., Kim, S., Wilson, C. J., Lehar, J., Kryukov, G. V., Sonkin, D., Reddy, A., Liu, M., Murray, L., Berger, M. F., Monahan, J. E., Morais, P., Meltzer, J., Korejwa, A., Jane-Valbuena, J., Mapa, F. A., Thibault, J., Brie-Furlong, E., Raman, P., Shipway, A., Engels, I. H., Cheng, J., Yu, G. K., Yu, J., Aspesi, P., de Silva, M., Jagtap, K., Jones, M. D., Wang, L., Hatton, C., Palescandolo, E., Gupta, S., Mahan, S., Sougnez, C., Onofrio, R. C., Liefeld, T., MacConaill, L., Winckler, W., Reich, M., Li, N., Mesirov, J. P., Gabriel, S. B., Getz, G., Ardlie, K., Chan, V., Myer, V. E., Weber, B. L., Porter, J., Warmuth, M., Finan, P., Harris, J. L., Meyerson, M., Golub, T. R., Morrissey, M. P., Sellers, W. R., Schlegel, R. & Garraway, L. A. The Cancer Cell Line Encyclopedia enables predictive modelling of anticancer drug sensitivity. Nature 483, 603-307, (2012).

4 Menden, M. P., Iorio, F., Garnett, M., McDermott, U., Benes, C. H., Ballester, P. J. & Saez-Rodriguez, J. Machine Learning Prediction of Cancer Cell Sensitivity to Drugs Based on Genomic and Chemical Properties. PLoS ONE 8, (2013).

5 Cohen, P. Protein kinases—the major drug targets of the twenty-first century? Nat Rev Drug Discov 1, 309-315, (2002).

6 Fabian, M. A., Biggs, W. H., Treiber, D. K., Atteridge, C. E., Azimioara, M. D., Benedetti, M. G., Carter, T. A., Ciceri, P., Edeen, P. T., Floyd, M., Ford, J. M., Galvin, M., Gerlach, J. L., Grotzfeld, R. M., Herrgard, S., Insko, D. E., Insko, M. A., Lai, A. G., Lelias, J.-M., Mehta, S. A., Milanov, Z. V., Velasco, A. M., Wodicka, L. M., Patel, H. K., Zarrinkar, P. P. & Lockhart, D. J. A small molecule-kinase interaction map for clinical kinase inhibitors. Nat Biotech 23, 329-336, (2005).

7 Karaman, M. W., Herrgard, S., Treiber, D. K., Gallant, P., Atteridge, C. E., Campbell, B. T., Chan, K. W., Ciceri, P., Davis, M. I., Edeen, P. T., Faraoni, R., Floyd, M., Hunt, J. P., Lockhart, D. J., Milanov, Z. V., Morrison, M. J., Pallares, G., Patel, H. K., Pritchard, S., Wodicka, L. M. & Zarrinkar, P. P. A quantitative analysis of kinase inhibitor selectivity. Nature Biotechnology 26, 127-132, (2008).

8 Anastassiadis, T., Deacon, S. W., Devarajan, K., Ma, H. & Peterson, J. R. Comprehensive assay of kinase catalytic activity reveals features of kinase inhibitor selectivity. Nat Biotechnol 29, 1039-1045, (2011).

9 Agarwal, A., Meckenzie, R. J., Carey, A., Davare, M., Eide, C. A., Watanabe-Smith, K., Braziel, R. M., Tyner, J. W., Bagby, G. C. & Druker, B. J. Critical Role Of Interleukin Receptor Signaling In Acute Myeloid Leukemia Identified Using An RNAi Functional Screen. Blood 122, 473-473, (2013).

10 Xu, C. C., Wu, L. M., Sun, W., Zhang, N., Chen, W. S. & Fu, X. N. Effects of TGF-beta signaling blockade on human A549 lung adenocarcinoma cell lines. Mol Med Rep 4, 1007-1015, (2011).

11 Wu, A. B., Wu, B., Guo, J. S., Luo, W. R., Wu, D., Yang, H. L., Zhen, Y., Yu, X. L., Wang, H., Zhou, Y., Liu, Z., Fang, W. Y. & Yang, Z. X. Elevated expression of CDK4 in lung cancer. J Transl Med 9, (2011).

12 Brabender, J., Danenberg, K. D., Metzger, R., Schneider, P. M., Park, J. M., Salonga, D., Holscher, A. H. & Danenberg, P. V. Epidermal growth factor receptor and HER2-neu mRNA expression in non-small cell lung cancer is correlated with survival. Clin Cancer Res 7, 1850-1855, (2001).

13 Li, C., Zhang, X., Cheng, L., Dai, L., Xu, F., Zhang, J., Tian, H., Chen, X., Shi, G., Li, Y., Du, T., Zhang, S., Wei, Y. & Deng, H. RNA interference targeting human FAK and EGFR suppresses human non-small-cell lung cancer xenograft growth in nude mice. Cancer Gene Therapy 20, 101+, (2013).

14 Camus, S., Quevedo, C., Menendez, S., Paramonov, I., Stouten, P. F. W., Janssen, R. A. J., Rueb, S., He, S., Snaar-Jagalska, B. E., Laricchia-Robbio, L. & Belmonte, J. C. L Identification of phosphorylase kinase as a novel therapeutic target through high-throughput screening for anti-angiogenesis compounds in zebrafish. Oncogene 31, 4333-4342, (2012).

15 Anastassiadis, T., Deacon, S. W., Devarajan, K., Ma, H. C. & Peterson, J. R. Comprehensive assay of kinase catalytic activity reveals features of kinase inhibitor selectivity. Nat Biotechnol 29, 1039-U1117, (2011).

16 Benjamini, Y. & Hochberg, Y. Controlling the false discovery rate: a practical and powerful approach to multiple testing. Journal of the Royal Statistical Society. Series B (Methodological), 289-300, (1995).

17 Tibshirani, R. Regression shrinkage and selection via the lasso. Journal of the Royal Statistical Society. Series B (Methodological), 267-288, (1996).

18 Zou, H. & Hastie, T. Regularization and variable selection via the elastic net. Journal of the Royal Statistical Society: Series B (Statistical Methodology) 67, 301-320, (2005).

19 Kohavi, R. in International joint Conference on artificial intelligence. 1137-1145 (Lawrence Erlbaum Associates Ltd, 1995).

20 Feala, J. D., Cortes, J., Duxbury, P. M., McCulloch, A. D., Piermarocchi, C. & Paternostro, G. Statistical Properties and Robustness of Biological Controller-Target Networks. PLoS ONE 7, e29374, (2012).

21 De Smet, R. & Marchal, K. Advantages and limitations of current network inference methods. Nature Reviews Microbiology 8, 717-729, (2010).

22 Marbach, D., Prill, R. J., Schaffter, T., Mattiussi, C., Floreano, D. & Stolovitzky, G. Revealing strengths and weaknesses of methods for gene network inference. Proceedings of the National Academy of Sciences 107, 6286, (2010).

23 Marbach, D., Costello, J. C., Kuffner, R., Vega, N. M., Prill, R. J., Camacho, D. M., Allison, K. R., Kellis, M., Collins, J. J., Stolovitzky, G. & Consortium, D. Wisdom of crowds for robust gene network inference. Nature Methods 9, 796-+, (2012).

24 Bar-Joseph, Z., Gerber, G. K., Lee, T. I., Rinaldi, N. J., Yoo, J. Y., Robert, F., Gordon, D. B., Fraenkel, E., Jaakkola, T. S. & Young, R. A. Computational discovery of gene modules and regulatory networks. Nat Biotechnol 21, 1337-1342, (2003).

25 Lemmens, K., De Bie, T., Dhollander, T., De Keersmaecker, S. C., Thijs, I. M., Schoofs, G., De Weerdt, A., De Moor, B., Vanderleyden, J. & Collado-Vides, J. DISTILLER: a data integration framework to reveal condition dependency of complex regulons in Escherichia coli. Genome Biol 10, R27, (2009).

26 Reiss, D., Baliga, N. & Bonneau, R. Integrated biclustering of heterogeneous genome-wide datasets for the inference of global regulatory networks. BMC bioinformatics 7, 280, (2006).

27 http://clinicaltrials.gov. <http://clinicaltrials.gov/show/NCT01291017> (2014).

28 Kawazu, M., Ueno, T., Kontani, K., Ogita, Y., Ando, M., Fukumura, K., Yamato, A., Soda, M., Takeuchi, K., Miki, Y., Yamaguchi, H., Yasuda, T., Naoe, T., Yamashita, Y., Katada, T., Choi, Y. L. & Mano, H. Transforming mutations of RAC guanosine triphosphatases in human cancers. P Natl Acad Sci USA 110, 3029-3034, (2013).

29 Potiron, V. A., Roche, J. & Drabkin, H. A. Semaphorins and their receptors in lung cancer. Cancer Lett 273, 1-14, (2009).

30 Feala, J. D., Cortes, J., Duxbury, P. M., Piermarocchi, C., McCulloch, A. D. & Patemostro, G. Systems approaches and algorithms for discovery of combinatorial therapies. Wires Syst Biol Med 2, 181-193, (2010).

31 Pedregosa, F., Varoquaux, G. 1., Gramfort, A., Michel, V., Thirion, B., Grisel, O., Blondel, M., Prettenhofer, P., Weiss, R. & Dubourg, V. Scikit-learn: Machine learning in Python. The Journal of Machine Learning Research 12, 2825-2830, (2011).

What is claimed is:

1. A method for quantitatively predicting effectiveness of drug combinations with two or more compounds for use as a therapy for a disease, optionally a cancer, the method comprising:
a) providing a biological sample from a patient suffering from a disease, optionally a cancer, the sample comprising cells from the patient;
b) providing a library of drugs in which each drug i has known residual activity A_{k,i} of each drug-target protein k under the effect of the drug i;
c) assaying the cells from the biological sample with each drug i to obtain a viability parameter vᵢ for each drug, wherein the assay is an endpoint assay that determines growth, survival, or death of a living cell;
d) performing a regression analysis from assay results to determine a training set and to identify residual activity parameters as predictors of the viability, wherein the results are modeled assuming a dependence according to Equation (I)
$${v}_{i} = {e}^{{β}_{0}} {\prod }_{k = 1}^{p} {( {A}_{k , i} )}^{{β}_{p}} \qquad ( I )$$
and the regression analysis provides a set of coefficients β₀, . . . , βₚ, where p is a number of drug-target proteins;
e) predicting the viability of a combination COMB with N drugs and dosages d=(d₁, d₂, . . . , d_{N}) as set forth in Equation (II)
$${v}_{COMB} = {e}^{{β}_{0}} {\prod }_{k = 1}^{p} {( {A}_{k , COMB} )}^{{β}_{p}} \qquad ( II )$$
where residual activity A_{k,COMB} of the drug target k under the effect of a combination with N drugs and dosages d=(d₁, d₂, . . . , d_{N}) is calculated as Equation (III)
$${A}_{k , COMB} = {\prod }_{j = 1}^{N} {( {A}_{k , j} )}^{{d}_{j}} ; \qquad ( III )$$
f) searching within a space of dosages d=(d₁, d₂, . . . , d_{N}) to find a corresponding optimal value of a predicted combination viability v_{COMB}; and
g) selecting the identified optimal combination of drugs as a combinatorial therapy for a patient having a same cancer or same disease.

2. The method of claim 1, wherein the equation in claim 1 (d) is reduced to a linear form using a logarithmic transformation and the coefficients (β₀, β₁, . . . , βₚ) are obtained using a linear regression procedure including the lasso, ridge, or elastic net methods.

3. The method of claim 1, wherein the biological sample comprises non-cancerous cells from different tissues for predicting toxicity of a combination .of two or more compounds.

4. The method of claim 3, wherein the predictions of viability and toxicity are combined in a prediction of the therapeutic index of a drug combination with two or more compounds.

5. The method of claim 1, wherein the predictions of viability are combined to predict synergistic and antagonistic properties of drug combinations.

6. The method of claim 1, wherein the magnitude of the coefficients β₁, . . . , βₚ is used to identify drug-target proteins whose inhibition or stimulation is associated to a positive therapeutic response.

7. The method of claim 1, wherein the library of drugs consists of compounds in the drug class of kinase inhibitors.
